(19) United States
(12) Patent Application Publication
(10) Pub. No.: US 2010/0260772 A1
(43) Pub. Date: Oct. 14, 2010

(54) METHODS FOR TREATING OR PREVENTING DISEASES ASSOCIATED WITH LOW BONE MASS

(75) Inventor: Gerard Karsenty, New York, NY (US)

Correspondence Address: KENYON & KENYON LLP, ONE BROADWAY, NEW YORK, NY, 10004, US

(73) Assignee: THE TRUSTEES OF COLUMBIA UNIVERSITY IN THE CITY OF NEW YORK, New York, NY (US)

(21) Appl. No.: 12/680,391

(22) PCT Filed: Sep. 26, 2008
(86) PCT No.: PCT/US08/77870

§ 371 Date: Apr. 26, 2010

Related U.S. Application Data
(60) Provisional application No. 60/976,403, filed on Sep. 28, 2007.

(51) Int. Cl. A61K 39/395; A61K 31/12; A61K 31/137; A61K 31/495; A61K 31/496; A61K 31/497; A61P 19/00; A61P 19/10

(52) U.S. Cl. 424/158.1; 424/172.1; 514/689; 514/646; 514/255.03; 514/253.04; 514/253.06; 514/252.11; 514/254.05

(57) ABSTRACT

Methods of treating or preventing diseases associated with low bone mass in a mammal in need of such treatment or prevention comprising administering to the mammal a therapeutically effective amount of an agent that increases tryptophan hydroxylase 2 activity or of an agonist of the brain serotonin HT2C receptor. Diseases associated with low bone mass include osteoporosis, osteopenia, Paget's disease, osteomalacia, and renal osteodystrophy.

This invention was made with U.S. government support under an NIH 2 R01 DK5883 grant. The government therefore has certain rights in the invention.

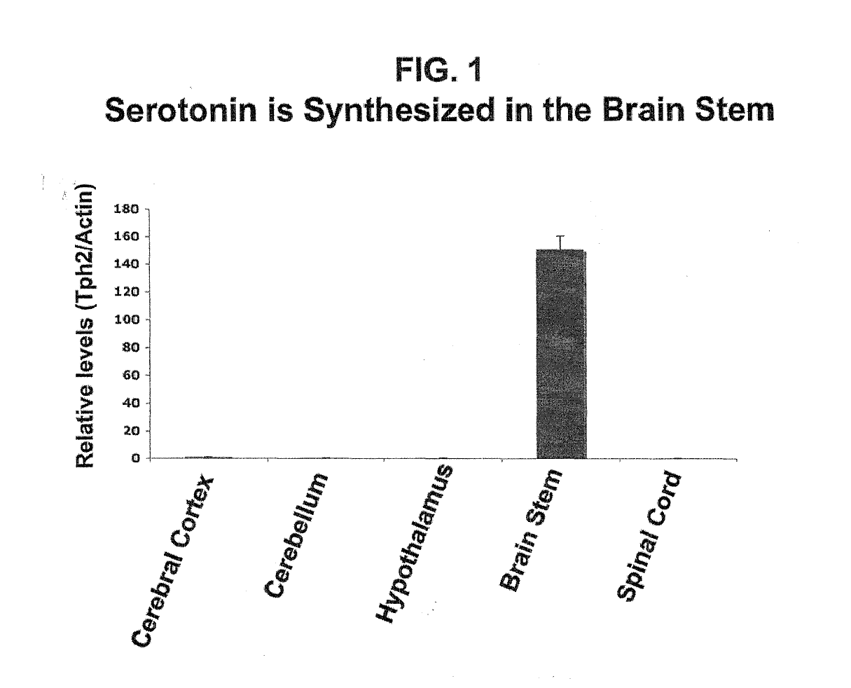

We have discovered that brain-derived serotonin causes an increase bone mass accrual, therefore bone mass is at least in part regulated through the central nervous system. As will be described below, elevated levels of brain serotonin are also associated with decreased sympathetic tone. Brain-derived serotonin (BDS) is synthesized exclusively in neurons of the brainstem in a two step biochemical pathway requiring tryptophan as a substrate and a brain-specific enzyme tryptophan hydroxylase 2 (Tph2), which enzyme is located exclusively in the brain. FIG. 1 Transgenic BDS knockout mice (lacking the enzyme Tph2 that synthesizes serotonin in brain) show low bone mass. We have discovered also that serotonin acts on the hypothalamus (VMH) via the brain receptor HT2C.

Certain embodiments of the invention are directed to a method of treating a bone disease comprising: administering to a mammal in need of said treatment a therapeutically effective amount of an agonist of the brain serotonin HT2C receptor, wherein the bone disease is characterized by a decreased bone mass relative to that of corresponding non-diseased bone. The agonist includes HT2C receptor agonists that are set forth in U.S. Pat. No. 6,403,651, the entire contents of which are hereby incorporated by reference. Bone diseases that can be treated according to the present invention include diseases associated with low bone mass including osteoporosis, osteopenia, Paget's disease, osteomalacia and renal osteodystrophy. Other embodiments are directed to therapeutic methods for treating or preventing bone diseases associated with low bone mass by administering a therapeutically effective amount of a compound that increases the activity of the serotonin synthesizing enzyme Tph2 that is localized in brain, or of serotonin reuptake blockers that target the brain with higher affinity than the periphery. In yet other embodiments a beta 2 adrenergic receptor blocker is administered to reduce sympathetic tone and increase bone mass, either alone or in combination with one or more of the following: an HT2C agonist, a BDS reuptake blocker, a leptin antagonist, and a leptin receptor blocker.

In certain embodiments the HT2C receptor agonist, a compound that increases Tph2 activity, a selective BDS reuptake blocker, or beta-2 adrenergic receptor blocker are administered together in a single preparation, or in different preparations at different times on the same day or sequentially over a period of time. In other embodiments one or more of these compounds are combined with a leptin antagonist and/or a leptin receptor blocker for therapeutic use.

Certain embodiments are further directed to the novel pharmaceuticals described herein including: a pharmaceutical comprising one or more HT2C receptor agonists for use in treating or preventing a bone disease associated with low bone mass, or for increasing bone mass in a mammal in need of such treatment; a pharmaceutical comprising one or more HT2C receptor agonists plus a leptin antagonist and/or a leptin ObRb receptor blocker, for use in treating or preventing a bone disease associated with low bone mass. Other embodiments include various combinations of an HT2C agonist, a beta-2 receptor blocker, a BDS reuptake blocker, an activator of Tph2, a leptin antagonist, and an ObRb receptor blocker.

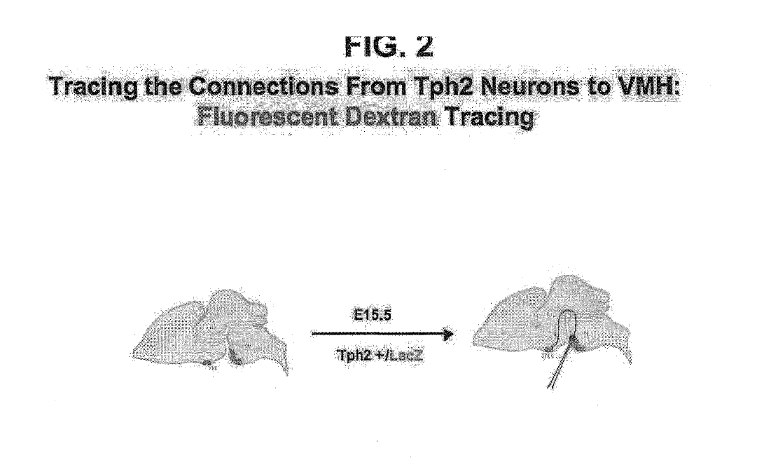

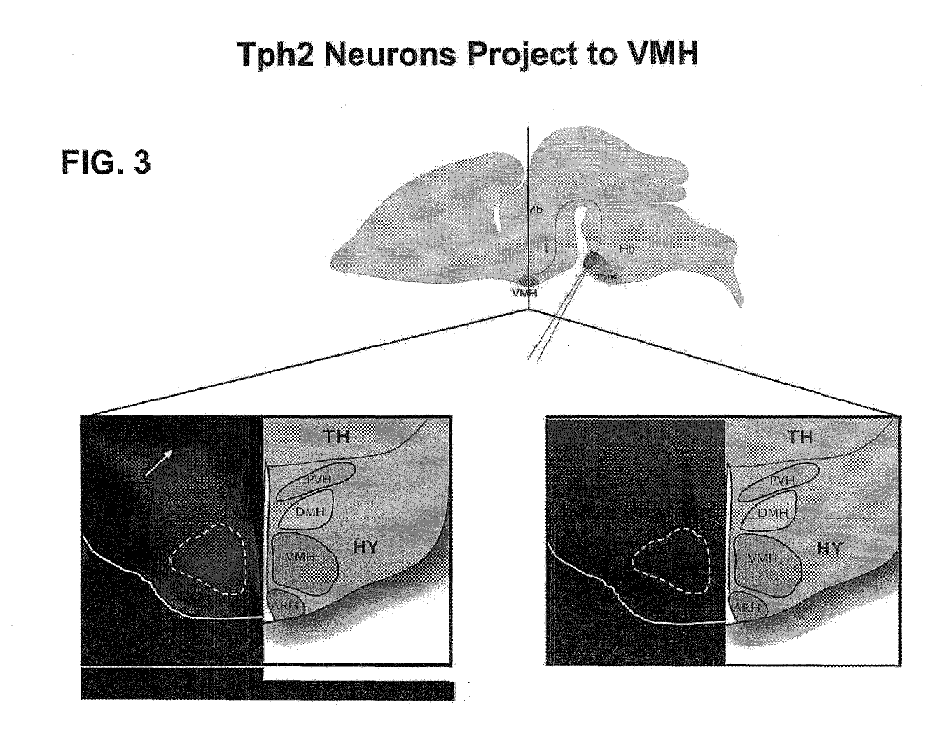

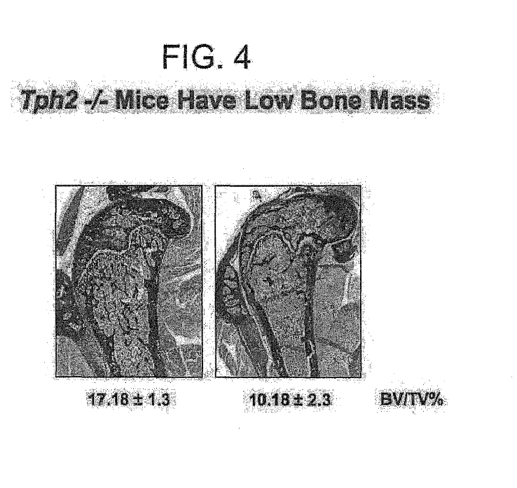

The experiments described below reveal not only a major role for BDS in bone remodeling but also an unexpectedly and totally novel mechanism whereby leptin regulates bone mass. Serotonin neurons project to several areas of the brain to regulate multiple functions such as cognition, behavior, appetite and others. Fluorescent dextran tracing showed that serotonergic neurons project from the brain stem to the ventromedial hypothalamus (VMH). FIG. 2 and FIG. 3. We discovered that our Tph2 knock out mice present as their main phenotype a severe low bone mass phenotype, thus identifying BDS as the first neuropeptide favoring bone mass accrual. FIG. 4.

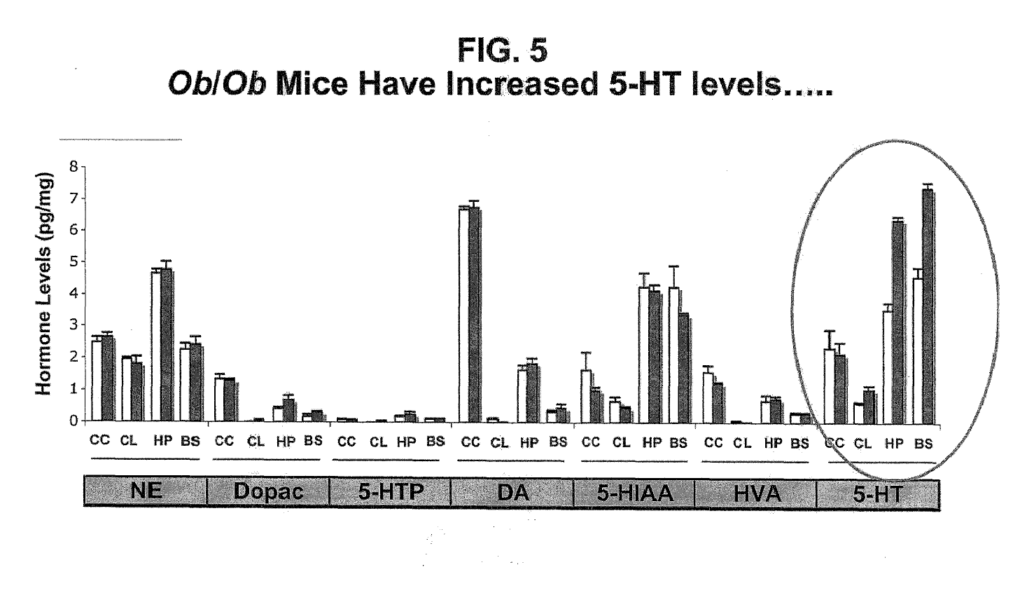

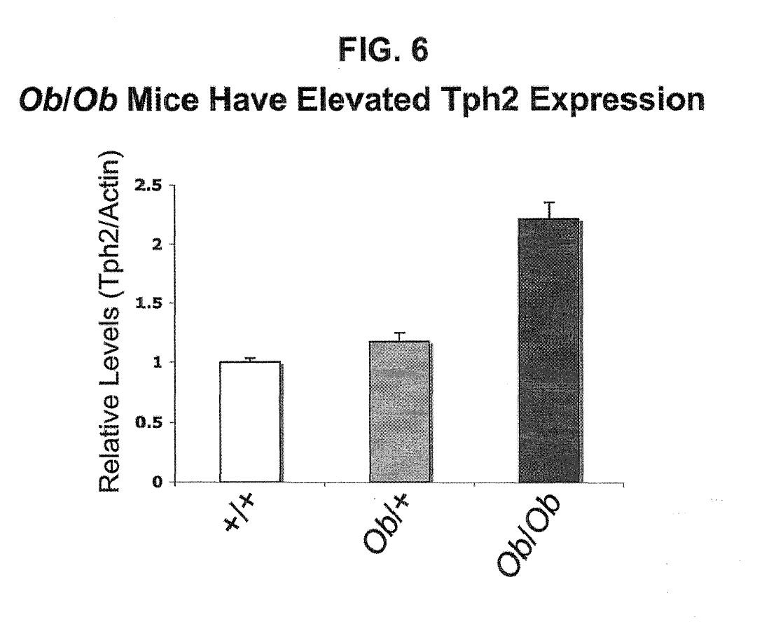

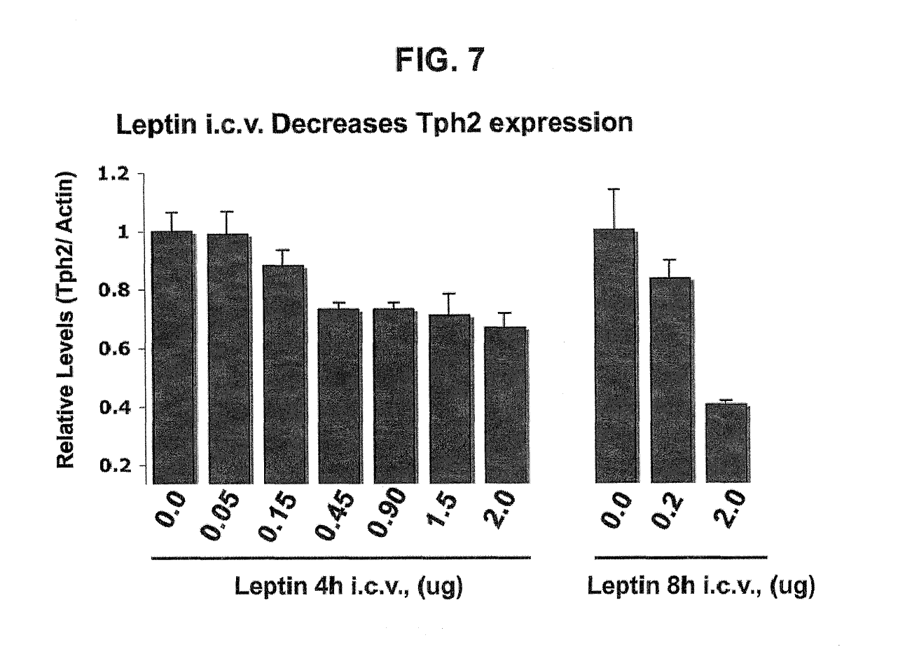

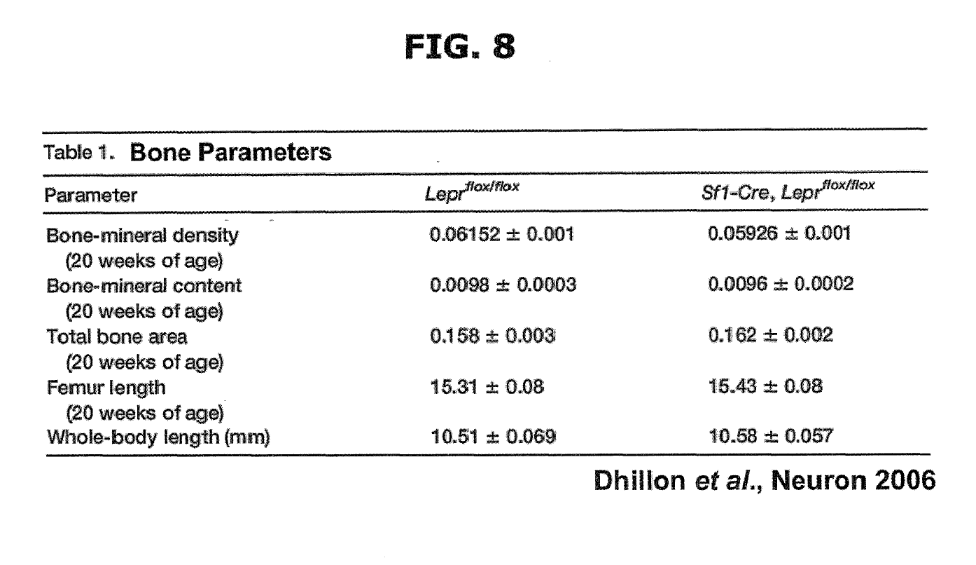

We now know that instead of the broadly accepted model where leptin acts on the hypothalamus, our data show the following model: leptin first acts on serotonergic neurons of the brain stem to inhibit synthesis of BDS whose biological function is to act on hypothalamic neurons via the a brain-specific receptor HT2C, thereby decreasing sympathetic tone and increasing bone mass. Importantly, BDS does not cross the blood brain barrier, dissociating the effects of serotonin in the brain from those in the periphery. Specifically, the experiments described below show that: 1) Leptin regulates Tph2 expression in brain stem: FIG. 5 and FIG. 6 show that leptin knockouts have increased serotonin (5HT) levels and increased Tph2 expression, and FIG. 7 shows that ICV injection of leptin decreases Tph2 expression in brain stem. 2) The Tph2−/− mice show a low bone mass phenotype that is due to a concomitant decrease in bone formation and increase in bone resorption parameters, which is exactly the opposite of what is seen in mice lacking the (32 adrenergic receptor. 3) Finally, sympathetic activity is increased in Tph2−/− mice. FIGS. 8-11.

Serotonin Biology

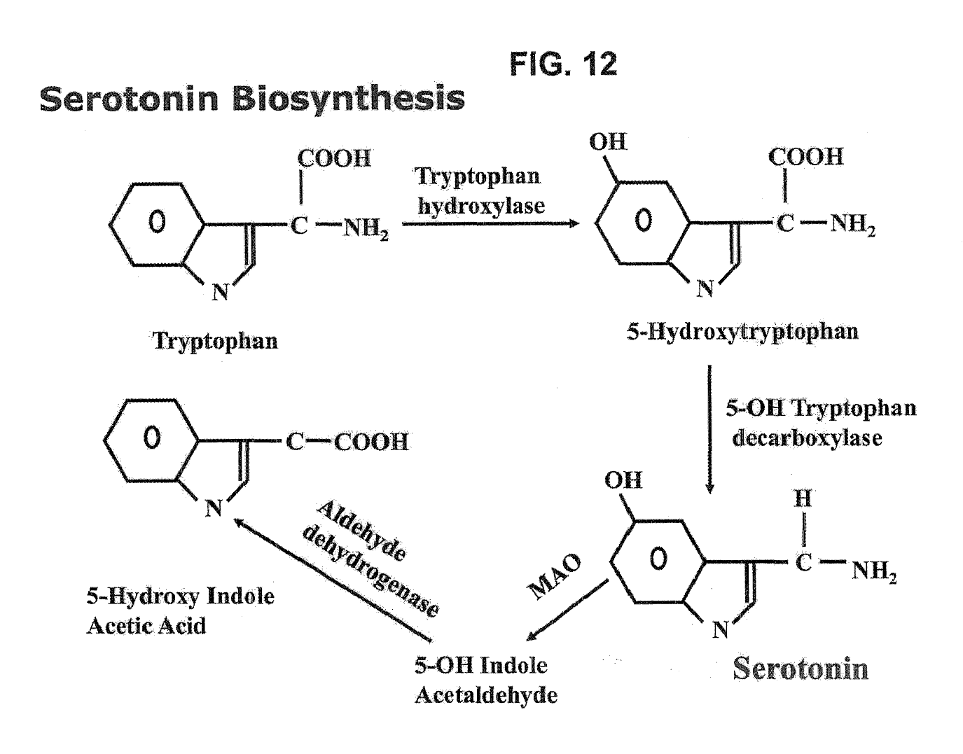

Serotonin or 5-hydroxytriptamine (5-HT) is a biogenic amine that functions as both a neurotransmitter and as a hormone in the mammalian central nervous system (CNS) and in the periphery. Serotonin is synthesized in two steps from the essential amino acid tryptophan. The first of these two steps is hydroxylation of tryptophan, a reaction carried out by two distinct enzymes in the CNS and in the periphery. In the CNS the enzyme is tryptophan hydroxylase 2 (Tph2); in the periphery the enzyme is tryptophan hydroxylase 1 (Tph1). FIG. 12. Those two enzymes are encoded by two different genes; moreover serotonin does not cross the blood brain barrier. Because of these features it is accurate to assume that, functionally, central and peripheral serotonin are two separate and distinct molecules that affect separate and distinct areas of the body.

Serotonin was first identified in the mucosa and was called enteramine. In humans, 90% of the total amount of serotonin in the body is present in GI enterochromaffin cells (90%); 5% in the brain. BDS is synthesized by Tph2-expressing neurons that are localized only in the brainstem. Serotonergic neurons projects then to several areas of the brain and the spinal cord. Once released from a neuron whether in the brain or in the periphery a reuptake mechanism deactivates the serotonin. In this mechanism, serotonin is taken up by the target cells or neurons it stimulates through the action of the enzyme 5 hydroxytryptamine transporter (5HTT), which is broadly expressed in the brain and peripheral tissues. BDS has been reported to regulate cognitive functions, appetite, perception, motor and sensory functions, affect and sleep. As a result most drugs used for the treatment of depression and other psychiatric disorders act by inhibiting the serotonin reuptake mechanism, and are thus called serotonin reuptake inhibitors (SSRI). The ability of BDS to influence so many functions relies in large part on the large number (14 receptor types, arranged in seven classes), the molecular diversity and the different pattern of expression of the various serotonin receptors.

The study of several transgenic animal models each lacking one specific serotonin receptor (HT1B, 1A, 1D or 2C) has verified the involvement of serotonin in various behavior-related functions. However, to date there has been no report linking BDS to peripheral physiological functions such as bone remodeling, nor any report of mice that do not make any BDS, i.e. transgenic Tph2 knockout mice.

In the last 8 years our laboratory has demonstrated that leptin is overall a negative regulator of bone mass, and that this function of leptin occurs through a central relay in the brain. This contention is based on several types of experiments ranging from analysis of bone histology in ob/ob (leptin knockout) mice before and after intracerebroventricular (ICV) infusion of leptin; and genetic or chemical lesioning of hypothalamic neurons followed by bone histologic analysis. Our analysis focused on two hypothalamic nuclei, the ventromedial hypothalamic (VMH) and the arcuate (ARC) nuclei for two reasons. First these nuclei are loaded with neurons expressing the signaling form of the leptin receptor (ObRb) at its highest level; second it was generally assumed that leptin acts directly on hypothalamic neurons to mediate its various effects. However, in view of the new work reported here, we now know that these assumptions are not all correct.

We know that expression of the leptin receptor ObRb is not restricted to hypothalamic neurons and can be found in particular in serotonin-producing neurons of the brainstem that also express Tph2. Second, following leptin injection there is increased cfos expression in brain stems that reflects the functional action of leptin as well as decreased Tph2 expression in brain stem. Third selective genetic ablation of ObRb in neurons of the VMH, a nucleus whose physical integrity was until now thought to be necessary for leptin regulation of bone mass, does not affect bone mass as was expected under the old theory. This latter result means that leptin's first site of action in the brain is not, at least as far as controlling bone mass is concerned, the hypothalamus but rather the brainstem where serotonergic neurons are localized. Thus, our results show that leptin regulates synthesis of serotonin which then in turn acts on hypothalamic neurons to down-regulate sympathetic activity and up-regulate bone mass. Thus we show that serotonin is a key integrator of leptin signaling, which is medically important because we now have a way to modulate BDS synthesis to increase bone mass accrual.

### RESULTS

Brain Derived Serotonin (BDS) is the First Neuropeptide Identified that Positively Regulates Bone Remodeling

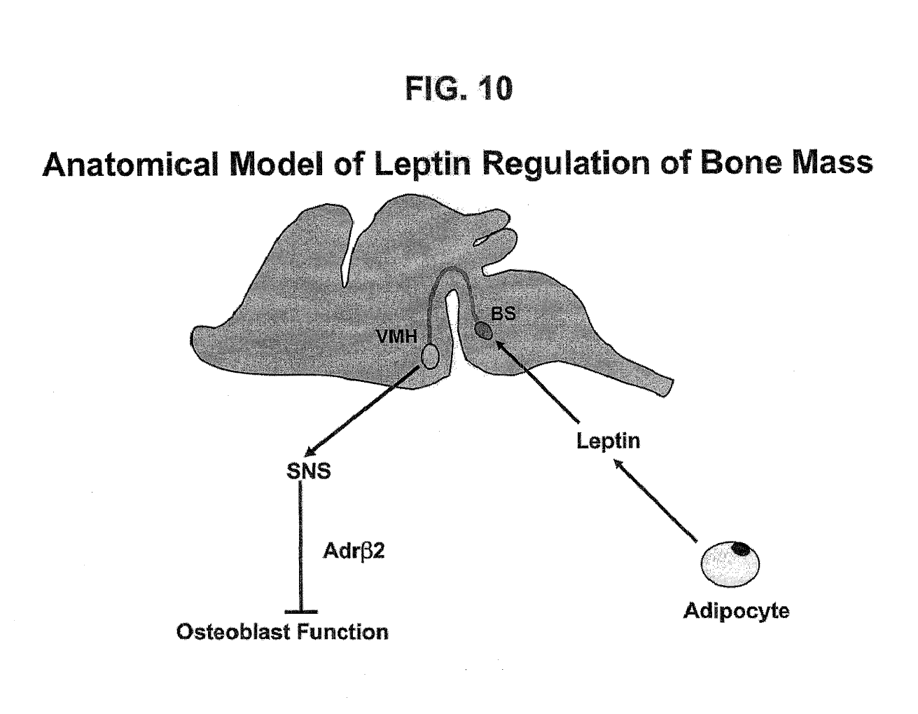

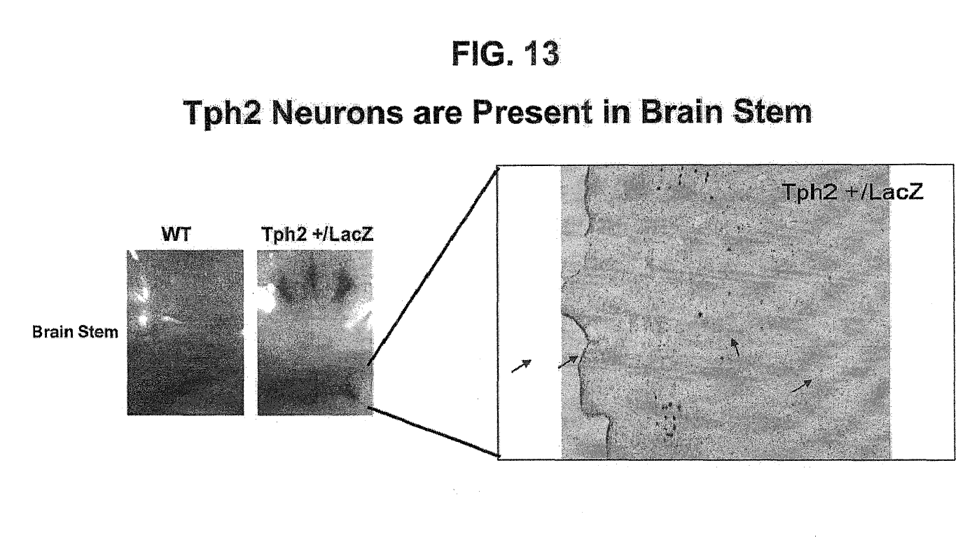

In the experiments described below, the cell-specific Tph2 in brainstem serotonergic neurons and HT2C, the serotonin receptor for BDS, were deleted using classical gene deletion procedures. Expression and function of Tph2 in mice was studied to determine the function of BDS. To accomplish this we inserted, through homologues recombination in embryonic stem (ES) cells, the LacZ gene in the Tph2 locus. See FIG. 13. Through β galactosidase staining we were able to identify Tph2-expressing neurons. This technique is particularly potent because of the high sensitivity of detection of β galactosidase staining. As shown in FIG. 1, Tph2-expressing neurons are restricted to the ventral raphe in the brainstem; we did not detect any expression of Tph2 outside the central nervous system. We also show using fluorescent dextran tracing that serotonergic neurons project from the brain stem to the ventromedial hypothalamus (VMH). FIG. 10.

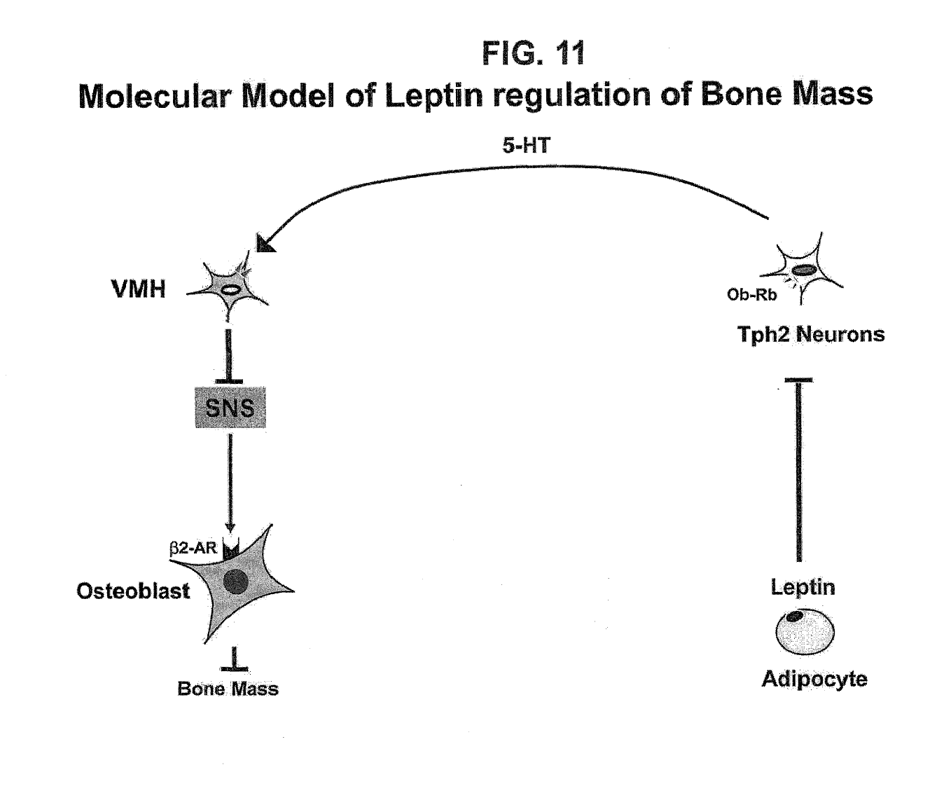

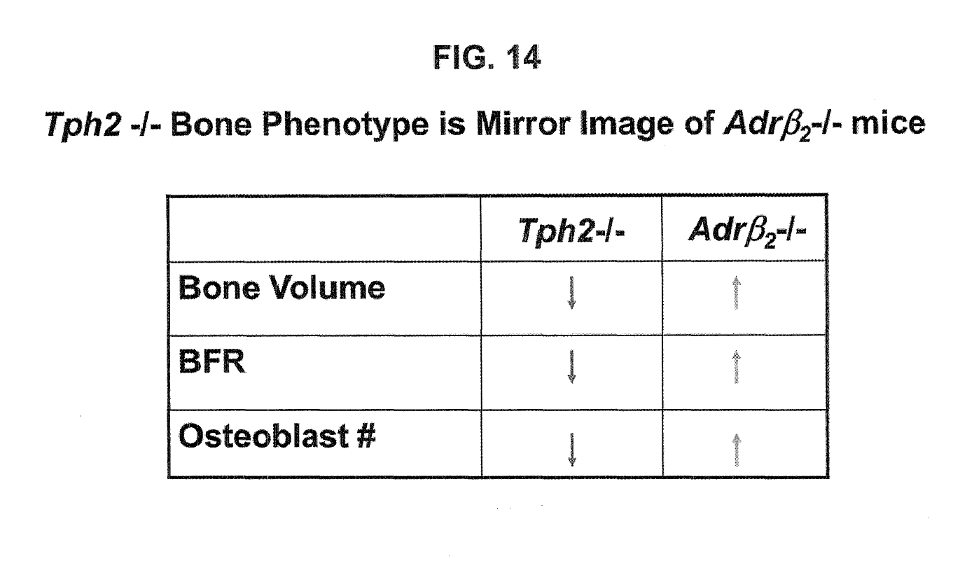

By generating mice homozygous for Tph2 deletion we were able to analyze the consequence of deleting this gene on bone remodeling and other homeostatic functions. We analyzed Tph2−/− mice at 6 and 12 weeks of age. At both stages Tph2 deletion resulted in a low bone mass phenotype that was secondary to an increase in bone resorption parameters and a decrease in bone formation. FIG. 8 and FIG. 14. Our results showed that there was no detectable serotonin in Tph2−/− brains; however, the blood concentration of serotonin was normal. This is consistent with the fact that BDS and gut-derived serotonin produced in primarily the duodenum (GDS) should be viewed at two completely different molecules physiologically, and that serotonin does not cross the blood brain barrier. The absence of serotonin in the brain of Tph2−/− knockouts not only affected bone mass accrual; there was also a decrease in food intake and body weight in Tph2−/− mice at 3 weeks of age. While this latter phenotype completely corrected itself and returned to normal levels by the time the mice were 6 weeks-old, bone remodeling abnormalities remained. Taken together this data indicates that BDS is a positive and powerful regulator of bone mass. It is in fact the first neuropeptide identified that positively regulates bone remodeling. More importantly the data define a completely novel model to explain leptin regulation of bone mass. According to our new model, leptin binds to its receptor ObRb on a Tph2-expressing serotonergic neuron, thereby inhibiting synthesis of serotonin. Under normal conditions BDS binds to HT2C-expressing neurons in the VMH, this in turn decreases sympathetic tone and increases bone mass. FIG. 11. Leptin stimulation reverses this result by decreasing the amount of serotonin binding to HT2C-expressing neurons in the VMH, leading to an increase in sympathetic activity and a decrease in bone mass.

Leptin Regulates Synthesis of BDS Through Its Regulation of Tph2.

We designed experiments to prove that leptin regulates Tph2 expression and/or serotonin synthesis. To show that the leptin receptor ObRb is expressed in Tph2-expressing, serotonergic neurons, we performed in situ hybridization for ObRb in Tph2+/− mice. Tph2-expressing neurons were labeled by β galactosidase because we had designed the mice so that LacZ was knocked-in the ObRb locus. In situ hybridization showed ObRb expression in Tph2-expressing neurons, although it was not limited to only serotonergic neurons, which is consistent with leptin affecting other physiologic properties than bone mass.

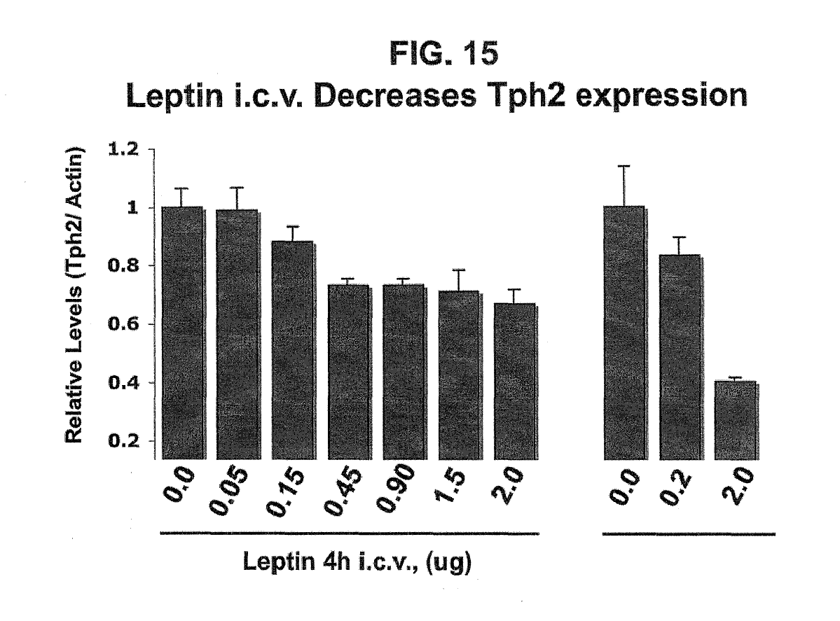

Next, we injected i.p 1 pg of leptin and studied its effects on Tph2 expression 4 and Ph. We also measured the brain concentration of serotonin in leptin knockouts (ob/ob mice). FIG. 5. The results showed that serotonin levels are increased in the brain stem and in the hypothalamus of leptin knockouts compared to controls. Leptin injection i.c.v. decreased Tph2 expression in brainstem by approximately 50%. FIG. 15. As an internal control we showed that the same leptin injection also decreased expression of neuropeptides Y (Npy), a leptin target gene in arcuate neurons. Measurement of serotonin content in various parts of the brain revealed that it is increased in hypothalamic and brainstem neurons in leptin knockouts compared to WT. FIG. 5. These results showed that leptin regulates Tph2 expression in the brainstem and that in absence of leptin there is an increase in serotonin hypothalamic content. Serotonin neurons in the brain stem have the ObRb receptors that bind to leptin, and they project to the hypothalamus. These results are consistent with our model described above.

In addition to treating or preventing the herein described bone diseases by administering compounds that increase brain serotonin or activate HT2C receptors, our results show that blocking leptin will increase levels of BDS. Therefore certain therapies are directed to drug combination therapy with agents that increase BDS or HT2C receptor activity, and leptin antagonists and/or ObRb receptor blockers. These drugs can be administered together in a single preparation, or in different preparations on the same day or sequentially on different days. Other embodiments are directed to novel pharmaceuticals comprising compounds that increase brain serotonin and/or activate HT2C receptors together with leptin antagonists and/or ObRb receptor blockers.

Sympathetic Activity Increases in the Absence of BDS

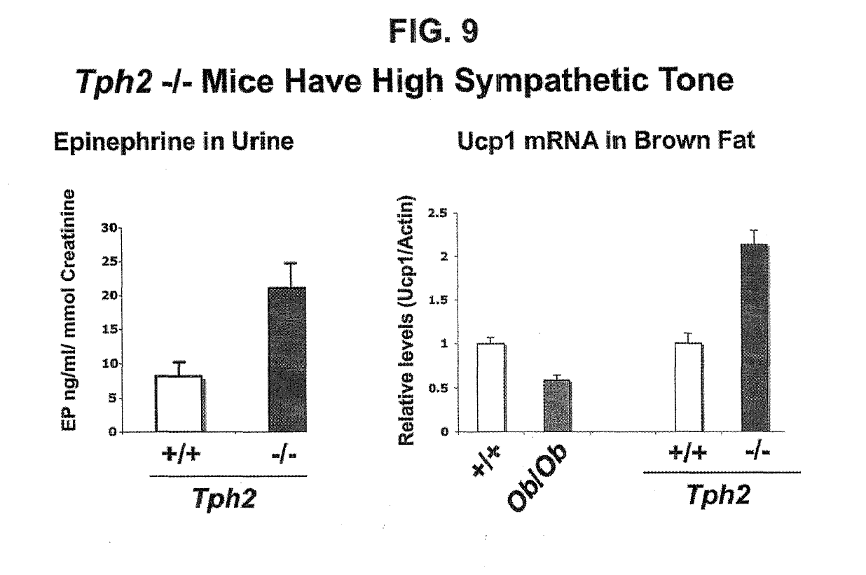

A second element of our model is that sympathetic activity is reduced by serotonin, resulting in increased bone mass. Remarkably the cellular abnormalities leading to the low bone mass phenotype in the Tph2−/− mice are the mirror image of what is seen in β2Ar−/− mice that lack sympathetic signaling in osteoblasts. FIG. 14. Measurement of sympathetic activity either by measuring uncoupling protein 1 (ucp1) expression in brown fat, or of epinephrine/norepinephrine urinary secretion showed that these two parameters were significantly increased in Tph2−/− mice as would be expected if sympathetic tone were increased. (FIG. 9). This increase in sympathetic signaling includes one molecular explanation for the low bone mass. It is also consistent with our working model showing that BDS under the control of leptin regulates bone mass accrual.

Based on this observation, certain embodiments of the invention are directed to methods for treating or preventing bone diseases associated with low bone mass by administering a compound that decreases sympathetic tone, preferably a beta-2 receptor antagonist, many of which are described in the art. Three new beta-2 specific blockers have been identified that can be used to reduce sympathetic tone and increase bone mass, alone or in combination with other drugs described herein; they are IPS339, ICI118,551, and Sandoz L1 32-468, Br J Ophthalmol. 1984 April; 68(4): 245-247. Butaxamine is also a bet2 blocker. Non-selective β antagonists include: metipranolol, nadol (a beta-specific sympatholytic which non-selectively blocks beta-2 adrenergic receptors); oxprenolol (a lipophilic beta blocker which passes the blood-brain barrier more easily than water soluble beta blockers), penbutolol, pindolol (a beta blocker that acts on serotonin (5-HT1A) receptors in the brain resulting in increased postsynaptic serotonin concentrations), and propranolol (known to readily cross the blood-brain barrier (BBB)), timolol and sotalol. The beta blockers can be administered together with agents that directly or indirectly increase BDS, including HT2C receptor agonists, agents that increase Tph2 activity or expression, and agents that specifically decrease reuptake of BDS.

Certain embodiments are also directed to new pharmaceuticals comprising agents that decrease sympathetic tone combined together with agents that increase BDS either directly (by activating or increasing the activity of Tph2) or indirectly by stimulating HT2C brain receptors. Leptin antagonists and ObRb receptor antagonists can also be included in these pharmaceuticals. Certain embodiments also include therapies for increasing bone mass in a patient having a bone disease associated with low bone mass by administering these drugs in a single preparation, in different preparations on the same day, or in different preparations on different days. For all of the therapies described herein, the administration of doses can be maintained until the desired result is achieved, and thereafter as needed to maintain the desired level of bone mass or the desired rate of bone formation.

HT2C Receptors in the VMH Signal Bone Remodeling

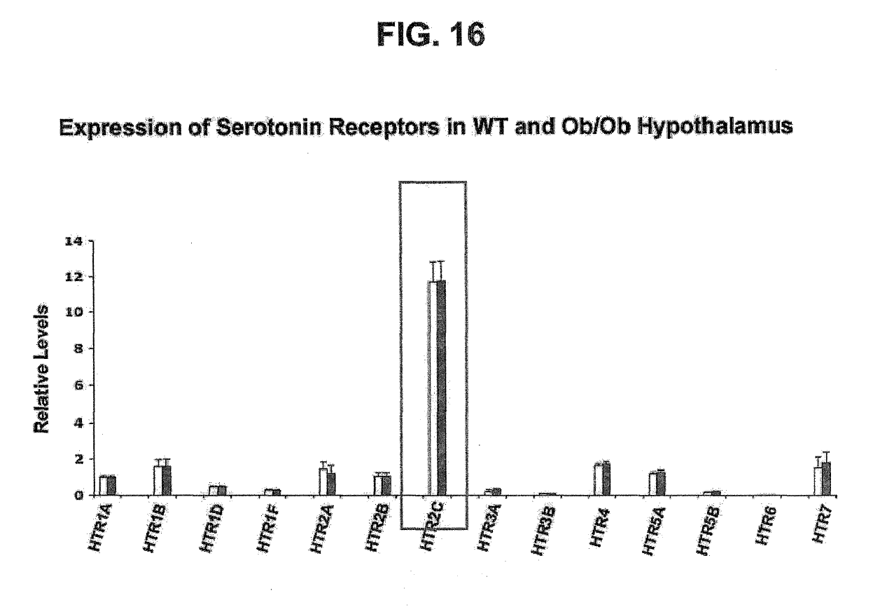

A third requirement of our working model is that there is one or more serotonin receptor expressed in neurons of the VMH nuclei to which the Tph2+ serotonergic neurons in the brain stem project thereby exerting their effect on sympathetic tone, and hence on bone mass accrual. To address this question we analyzed by real time PCR the expression of known serotonin receptors in the hypothalamus. FIG. 16. HT2C is the most highly expressed serotonin receptor in hypothalamus; furthermore, in situ hybridization showed that it is predominant if not restricted to VMH neurons. Moreover a broader tissue survey showed that HT2C is not expressed anywhere else in the brain, at a significant level, except for the cerebellum.

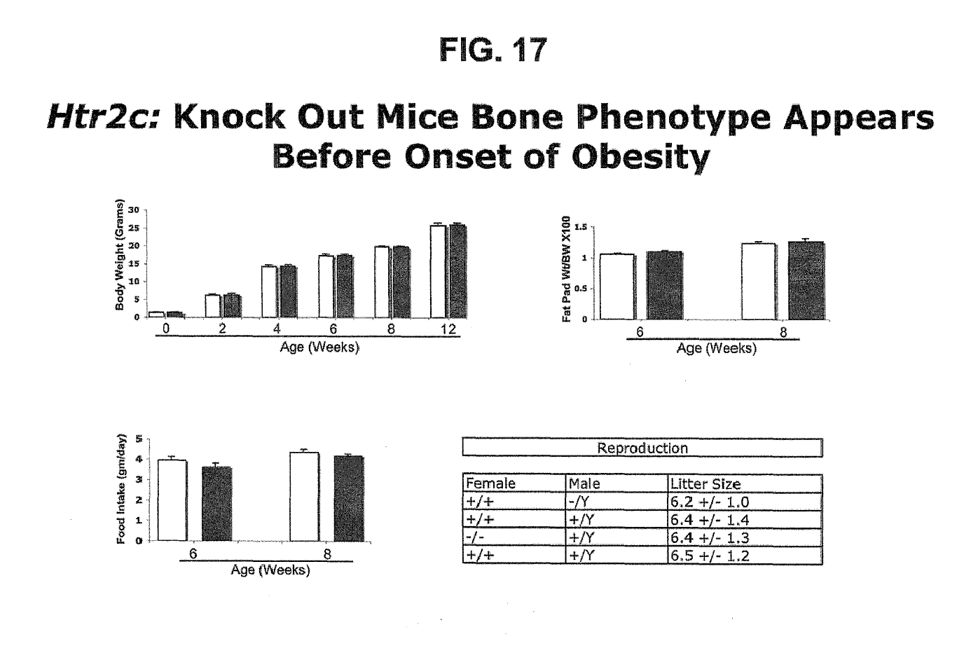

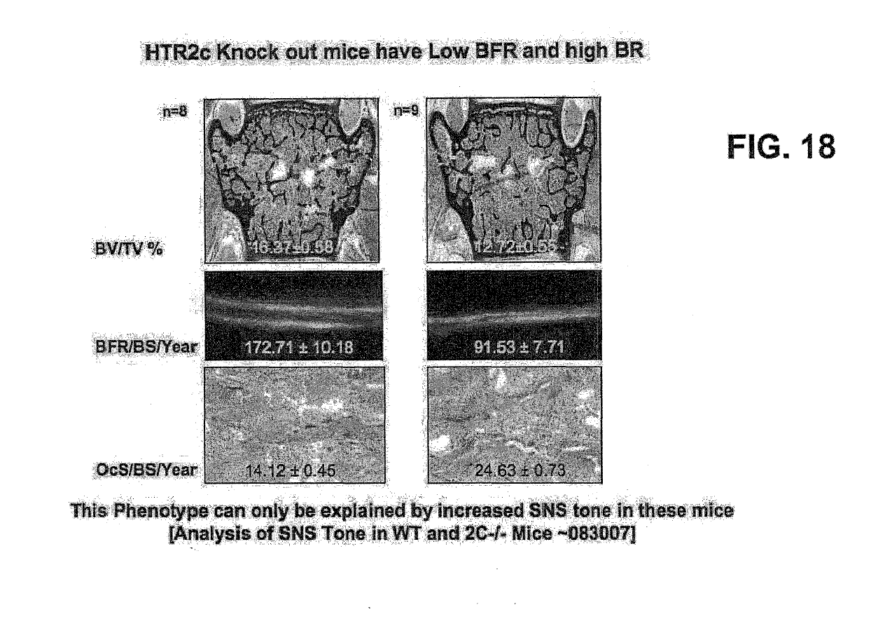

Data we obtained from Dr. L. Teacolt at UCSF for HT2C-deficient mice, show that at 8 weeks age Ht2c−/− mice have normal food intake levels (another function regulated by leptin) and normal body weight. FIG. 17 and FIG. 18. Further more, and this is highly relevant for our working hypothesis, we see that HT2C−/− mice display an increase in sympathetic activity at 8 weeks of age as was measured by Ucp1 expression in brown fat and urinary elimination of catecholamines. Taken together this data is consistent with a model linking serotonin signaling and sympathetic activity to bone mass regulation.

Others have identified HT2C-specific agonists which a high affinity for this serotonin binding site. U.S. Pat. No. 6,403,651, incorporated herein by reference. We have discovered that these agonists can be administered therapeutically to increase bone mass, thereby providing a much needed treatment for diseases such as osteoporosis. U.S. Pat. No. 6,403,651. Among the compound or compounds having a high affinity for the 5-HT2C serotonin receptor are the following, although it should be stressed that the invention is not limited to these compounds:

Among the agonists having a high affinity for the 5-HT2C serotonin receptor are the following, although it should be stressed that the invention is not limited to these compounds: (+/−)-1-(4-iodo-2,5-dimethoxy-phenyl)-2-aminopropane, (DOI); 1-(3-chlorophenyl)piperazine, (mCPP) and compounds which are metabolized to mCPP (desyrel, nefazodone and tradozone); 1-(alpha,alpha,alpha-trifluoro-m-tolyl)-piperazine, (TFMPP); (dl)-4-bromo-2,5-dimethoxyamphetamineHCl, (DOB); (dl)-2,5-dimethoxy-4-methylamphetamine HCl, (DOM); mesulergine; ritanserin; (clozapine; loxapine; R(+)-2-di-n-propylamino-8-hydroxy-1,2,3,4-tetrahydronapthalene, (SCH 23390); tiosperone; fluperlapine; rilapine; chlorpromazine; ketanserin; risperidone; cis-fluphenixol; quipazine; 6-chloro-2-(1-piperazinyl)pyrazine, (MK-212); spiperone; metergoline; methysergide; 6-methyl-1-(1-methylethyl)-ergoline-8-carboxylic acid (8.beta.)-2-hydroxy-1-methylpropyl ester(Z)-2-butenedioate(1:1), (LY-53857); methiothepin; cyproheptadine; perenpirone; N-(1-methyl-5-indolyl-N-(3-pyridyl) urea, (SB-200646); pitozifen; 2-(2-dimethylaminoethylthio)-3-phenylquinoline, (ICI-169-369); lisuride; methergine; piremperone; ergometrine.

Terminology

The following terms used herein shall have the meaning indicated:

Leptin, (“Ob”) as used herein, is defined by the endogenous polypeptide product of an ob gene, preferably a human ob gene, of which the known activities are mediated through the hypothalamus.

Leptin receptor (“ObRb”), as used herein, is defined by the receptor through which the leptin hormone binds to generate its signal; preferably, this term refers to a human leptin receptor.

Catecholamines, as used herein, is defined as being an endogenous amine-containing derivatives of catechol, 1,2-dihydroxybenzene, preferably norepinephrine and epinephrine.

Adrenergic receptor (“AR”), as used herein, is defined by the receptor through which catecholamines bind to generate its signal; preferably, this term refers to a human adrenergic receptor. One type of adrenergic receptor is the beta-2 adrenergic receptor.

NPY, as used herein, is defined as neuropeptide Y, preferably human neuropeptide Y. Neuropeptide Y (NPY) is a member of the pancreatic polypeptide family. It is to be understood that the term NPY, as used herein is intended to encompass not only neuropeptide Y but also its peptide relatives in the pancreatic polypeptide family, e.g., peptide YY (PYY), and pancreatic polypeptide (PP).

Ciliary neurotrophic factor (“CNTF”), as used herein, is defined by the endogenous polypeptide product of a CNTF gene, preferably a human CNTF gene, of which the known activities are mediated through the central and peripheral (including autonomous) nervous system.

Bone disease, as used herein, refers to any bone disease or state which results in or is characterized by loss of health or integrity to bone and includes, but is not limited to, osteoporosis, osteopenia, faulty bone formation or resorption, Paget's disease, fractures and broken bones, and bone metastasis. More particularly, bone diseases which can be treated and/or prevented in accordance with the present invention include bone diseases characterized by a decreased bone mass relative to that of corresponding non-diseased bone (e.g., osteoporosis, osteopenia and Paget's disease). Prevention of bone disease includes actively intervening as described herein prior to onset to prevent the disease. Treatment of bone disease encompasses actively intervening after onset to slow down, ameliorate symptoms of, or reverse the disease or situation. More specifically, treating, as used herein, refers to a method that modulates bone mass to more closely resemble that of corresponding non-diseased bone (that is a corresponding bone of the same type, e.g., long, vertebral, etc.) in a non-diseased state.

Leptin receptor antagonist, as used herein, refers to a factor which neutralizes or impedes or otherwise reduces the action or effect of a leptin receptor. Such antagonists can include compounds that bind leptin or that bind leptin receptor. Such antagonists can also include compounds that neutralize, impede or otherwise reduce leptin receptor output, that is, intracellular steps in the leptin signaling pathway following binding of leptin to the leptin receptor, i.e., downstream events that affect leptin/leptin receptor signaling, that do not occur at the receptor/ligand interaction level. Leptin receptor antagonists may include, but are not limited to proteins, antibodies, small organic molecules or carbohydrates, such as, for example, acetylphenol compounds, antibodies which specifically bind leptin, antibodies which specifically bind leptin receptor, and compounds that comprise soluble leptin receptor polypeptide sequences.

An agent is said to be administered in a “therapeutically effective amount” if the amount administered results in a desired change in the physiology of a recipient mammal, e.g., results in an increase in bone mass relative to that of a corresponding bone in the diseased state; that is, results in treatment, i.e., modulates bone mass to more closely resemble that of corresponding non-diseased bone (that is a corresponding bone of the same type, e.g., long, vertebral, etc.) in a non-diseased state.

Sequences of leptin and leptin receptor, including human leptin and leptin receptors, are well known. For a review of leptin receptor proteins, see, e.g., Friedman and Halaas, 1998, Nature, 395:763-770 and U.S. Pat. No. 5,972,621. For leptin sequences, including human leptin coding sequences and leptin gene regulatory sequences, see, e.g., Zhang et al., 1994, Nature 372:425-432; de la Brousse et al., 1996, Proc. Natl. Acad. Sci. USA 93:4096-4101; He et al., 1995, J. Biol. Chem. 270:28887-28891; Hwang et al., 1996, Proc. Natl. Acad. Sci. USA 93:873-877; and Gong et al., 1996, J. Biol Chem 271:3971-3974.

Sequences of adrenergic receptors, include human adrenergic receptors, are well known (see, e.g., U.S. Pat. Nos. 6,274,706; 5,994,506; 5,817,477; and 5,595,880).

Any technique known in the art may be used to introduce the transgenes described herein into animals or to “knock-out” or inactivate endogenous genes to produce the founder lines of transgenic animals. Such animals can be utilized as part of a screening methods of the invention, and cells and/or tissues from such animals can be obtained for generation of additional compositions (e.g., cell lines, tissue culture systems) that can also be utilized as part of the screening methods of the invention.

Techniques for generation of transgenic animals are well known to those of skill in the art and include, but are not limited to, pronuclear microinjection (Hoppe & Wagner, 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

Transgenic animals can carry the transgene in all their cells. Alternatively, such animals can carry the transgene or transgenes in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al., 1992, Proc. Natl. Acad. Sci. USA 89: 6232-6236. The regulatory sequences required for such a cell-type specific activation will depend upon the particular cell type of interest, and will be apparent to those of skill in the art. When it is desired that the transgene be integrated into the chromosomal site of the endogenous gene, gene targeting is preferred. Briefly, when such a technique is to be utilized, vectors containing some nucleotide sequences homologous to the endogenous gene of interest are designed for the purpose of integrating, via homologous recombination with chromosomal sequences, into and disrupting the function of the nucleotide sequence of the gene. The transgene may also be selectively introduced into a particular cell type, thus inactivating the endogenous gene in only that cell type, by following, for example, the teaching of Gu et al., 1994, Science 265: 103-106. The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR.

Alternatively, there is a method of diagnosing or prognosing a bone disease in a mammal, such as a human, comprising: (a) measuring serotonin levels in cerebrospinal fluid of a mammal, e.g., a mammal suspected of exhibiting or being at risk for the bone disease; and (b) comparing the level measured in (a) to the level in control cerebrospinal fluid, so that if the level obtained in (a) is lower than that of the control, the mammal is diagnosed as exhibiting or being at risk for the bone disease, wherein the bone disease is characterized by a decreased bone mass relative to that of corresponding non-diseased bone.

In accordance with another aspect of the present invention, there is a method of monitoring efficacy of a compound for treating a bone disease in a mammal, such as a human, comprising: (a) administering the compound to a mammal; (b) measuring serotonin levels in cerebrospinal fluid of the mammal; and (c) comparing the level measured in (b) to the level in cerebrospinal fluid of the mammal prior to administering the compound, thereby monitoring the efficacy of the compound on serotonin in csf (the desired effect being an increase in serotonin in csf), wherein the bone disease is characterized by a decreased bone mass relative to that of corresponding non-diseased bone. Preferred compounds are ones that increase BDS levels relative to that observed prior to administration. Alternatively, symptoms of the disease or other indicia of bone mass can be monitored to determine efficacy, such as bone density measurements, reduced indicia of bone degeneration, increased serum or dynamic markers of bone formation, etc.

The methods described herein may involve the measurement of sympathetic tone of a mammal, with the goal being to decrease sympathetic tone by administering beta-2 adrenergic blockers. Sympathetic tone relates to the relative state of activation or “tension” of the sympathetic nervous system. Sympathetic activity controls a variety of functions, and are measured in a number of ways, including, but not limited to, direct measurements of nerve firing rates (see, e.g., Esler & Kay, 2000, J. Cardiovasc. Pharmacol. 35(7 Suppl 4):S1-7), levels of plasma catecholamines (see, e.g., Urban et al., 1995, European Journal of Pharmacology, 282:29-37), variations in heat rate (see, e.g., Boutouyrie et al., 1994, Am. J. Physiol. 267 (4 Pt 2):H1368-76), or renal sympathetic nerve activity (see, e.g., Feng et al., 1992, Journal of Pharmacology and Experimental Therapeutics, 261:1129-1135).

In some cases, upon identifying a mammal (e.g., human) exhibiting low bone mass and/or lower levels of serotonin in cerebrospinal fluid relative to that of a corresponding control sample, the mammal is a candidate for treatment as described herein to increase BDS. Alternatively, if an animal has low serotonin in the csf, it can be considered to be at risk for developing disease.

Among the techniques well known to those of skill in the art for measuring bone mass are ones that include, but are not limited to, skeletal X-ray, which shows the lucent level of bone (the lower the lucent level, the higher the bone mass); classical bone histology (e.g., bone volume, number and aspects of trabiculi/trabiculations, numbers of osteoblast relative to controls and/or relative to osteoclasts); and dual energy X-ray absorptiometry (DEXA) (Levis & Altman, 1998, Arthritis and Rheumatism, 41:577-587) which measures bone mass and is commonly used in osteoporosis.

The methods described herein may further be used to diagnose individuals at risk for bone disease. Such individuals include, but are not limited to, peri-menopausal women (as used herein, this tern is meant to encompass a time frame from approximately 6 months prior to the onset of menopause to approximately 18 months subsequent to menopause) and patients undergoing treatment with corticosteroids, especially long-term corticosteroid treatment.

BFR means bone formation rate.

Immunoassays and non-immunoassays for serotonin or conserved variants or peptide fragments thereof will typically comprise incubating a sample, cerebrospinal fluid in the presence of a detectably labeled antibody capable of identifying serotonin, and detecting the bound antibody by any of a number of techniques well-known in the art.

Leptin antagonists also include agents, or drugs, which decrease, inhibit, block, abrogate or interfere with binding of leptin to its receptors or extracellular domains thereof; agents which decrease, inhibit, block, abrogate or interfere with leptin production or activation; agents which are antagonists of signals that drive leptin production or synthesis, and agents which prohibit leptin from reaching its receptor, e.g., prohibit leptin from crossing the blood-brain barrier. Such an agent can be any organic molecule that inhibits or prevents the interaction of leptin with its receptor, or leptin production (see, e.g., U.S. Pat. No. 5,866,547). Leptin antagonists include, but are not limited to, anti-leptin antibodies, receptor molecules and derivatives which bind specifically to leptin and prevent leptin from binding to its cognate receptor.

Examples of ObRb antagonists are acetylphenols, which are known to be useful as antiobesity and antidiabetic compounds. Since acetylphenols are antagonists of the ObR, they prevent binding of leptin (Ob) to the ObR. Thus, in view of the teachings of the present invention, the compounds would effectively cause an increase in bone mass. For specific structures of acetylphenols which can be used as ObR antagonists, see U.S. Pat. No. 5,859,051.

Additional antagonists of the ObRb, and other compounds that modulate ObR gene expression or ObR activity that can be used for diagnosis, drug screening, clinical trial monitoring, and/or the treatment of bone disorders can be found in U.S. Pat. Nos. 5,972,621; 5,874,535; and 5,912,123.

An adrenergic antagonist, as used herein, refers to a factor which neutralizes or impedes or otherwise reduces the action or effect of an adrenergic receptor. Such antagonists can include compounds that bind catecholamines or that bind adrenergic receptors. Such antagonists can also include compounds that neutralize, impede or otherwise reduce catecholamine receptor output, that is, intracellular steps in the adrenergic signaling pathway following binding of catecholamines to the adrenergic receptor, i.e., downstream events that affect adrenergic signaling, that do not occur at the receptor/ligand interaction level. Beta-2 adrenergic receptor antagonists are preferred for reducing sympathetic tone, thereby increasing bone mass.

Any method which neutralizes, slows or inhibits leptin (Ob) or (ObR) expression (either transcription or translation), levels, or activity can be used in combination with agonists of HT2C, agents that increase BDS or Tph2 activity), and beta blockers to treat or prevent a bone disease characterized by a decrease in bone mass relative to a corresponding non-diseased bone by effectuating an increase in bone mass. Antisense approaches involve the design of oligonucleotides (either DNA or RNA) that are complementary to Ob or ObR mRNA. The antisense oligonucleotides will bind to the complementary Ob or ObR mRNA transcripts and prevent translation.

Pharmaceutical Formulations and Methods of Treating Bone Disorders

The compounds of this invention can be formulated and administered to inhibit a variety of bone disease states by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of the compound sufficient to result in amelioration of symptoms of the bone disease and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

Dose determinations and formulations of the pharmaceutical compositions for use in accordance with the present invention are described in US Patent No. 20060165683, which is incorporated herein by reference in its entirety, or may be formulated in any conventional manner known in the art.

1. A method of treating or of preventing a disease associated with low bone mass or a symptom thereof comprising administering to a mammal in need thereof a therapeutically effective amount of an agent that increases Tph2 activity by contact of the agent with Tph2.

2. A method of treating or of preventing a disease associated with low bone mass or a symptom thereof comprising administering to a mammal in need thereof a therapeutically effective amount of an agent that decreases the uptake of brain derived serotonin.

3. A method of treating or of preventing a disease associated with low bone mass or a symptom thereof comprising administering to a mammal in need thereof a therapeutically effective amount of an HT2C receptor agonist.

4. The method as in one of claims 1-3 wherein the disease is osteoporosis.

5. A pharmaceutical composition for use in a mammal comprising a therapeutically effective amount of a beta-2 adrenergic antagonist and a member selected from the group consisting of an HT2C receptor agonist, an agent that increases Tph2 activity by contact of the agent with Tph2, an agent that decreases the uptake of brain derived serotonin, a leptin antagonist, and an ObR blocker.

6. A pharmaceutical composition for use in a mammal comprising a therapeutically effective amount of an HT2C receptor agonist and a member selected from the group consisting of a beta-2 adrenergic antagonist, an agent that increases Tph2 activity by contact of the agent with Tph2, an agent that decreases the uptake of brain derived serotonin, a leptin antagonist, and an ObR blocker.

7. A pharmaceutical composition for use in a mammal comprising a therapeutically effective amount of an agent that increases Tph2 activity by contact of the agent with Tph2 and a member selected from the group consisting of an HT2C receptor agonist, a beta-2 adrenergic antagonist, an agent that decreases the uptake of brain derived serotonin, a leptin antagonist, and an ObR blocker.

8. A pharmaceutical composition for use in a mammal comprising a therapeutically effective amount of an agent that decreases the uptake of brain derived serotonin and a member selected from the group consisting of an HT2C receptor agonist, a beta-2 adrenergic antagonist, a leptin antagonist, an agent that increases Tph2 activity by contact of the agent with Tph2, and an ObR blocker.

9. The pharmaceutical composition as in one of claims 5-8, wherein the leptin antagonist is selected from the group consisting of acetylphenol, an antibody that binds leptin, and an antibody that binds leptin receptor.

10. A method of increasing bone mass in a mammal comprising administering to a mammal in need thereof an agent that increases Tph2 activity by contact of the agent with Tph2 in an amount that increases BDS.

11. A method of increasing bone mass in a mammal comprising administering to a mammal in need thereof an agent that decreases the uptake of brain derived serotonin in an amount that increases BDS.

12. A method of increasing bone mass in a mammal comprising administering to a mammal in need thereof a therapeutically effective amount of an HT2C receptor agonist.

13. The method of claims 3 or 12, wherein the HT2C agonist is a member selected from the group consisting of: (+/−)-1-(4-iodo-2,5-dimethoxy-phenyl)-2-aminopropane; 1-(3-chlorophenyl)piperazine; desyrel; nefazodone; tradozone; 1-(alpha,alpha,alpha-trifluoro-m-tolyl)-piperazine; (dl)-4-bromo-2,5-dimethoxyamphetamineHCl; (dl)-2,5-dimethoxy-4-methylamphetamine HCl; quipazine; and 6-chloro-2-(1-piperazinyl)pyrazine.
